(19) United States
(12) Patent Application Publication
(10) Pub. No.: US 2010/0063403 A1
(43) Pub. Date: Mar. 11, 2010

(54) Therapeutic focused energy delivery

(76) Inventor: Avi Mendelson, Hinanit (IL)

Correspondence Address: FENNEMORE CRAIG, 3003 NORTH CENTRAL AVENUE, SUITE 2600, PHOENIX, AZ, 85012, US

(21) Appl. No.: 12/230,896

(22) Filed: Sep. 8, 2008

(51) Int. Cl. A61B 6/00; A61N 7/00

(52) U.S. Cl. 600/476; 601/2

(57) ABSTRACT

A method and an apparatus adapted to perform three dimensional (3D) position-tracking of a transducer unit comprising a three dimensional (3) target, the 3D target adapted to show a position of the transducer unit, and further adapted to facilitate tracking a tilt angle of the transducer unit; and an optical imager adapted to acquire a two dimensional (2D) target image of the target.

### FIELD

The invention relates to apparatus and methods for transmitting a therapeutic focused energy.

### BACKGROUND

Therapeutic focused energy delivery (TFED) is a non-invasive method commonly used in the medical field, both for diagnostic and therapeutic purposes. An ultrasound scanner is an example of one of the applications of TFED. By delivering focused ultrasound energy to a region of tissue, certain medical conditions may be diagnosed, such as tumors and renal stones. Ultrasound is also used for monitoring fetus development during pregnancy.

TFED may be used for ablation and/or destroying of pathogenic objects and various tissues. TFED may also selectively target and disrupt subcutaneous fat cells and adipose tissue in different parts of a body during non-invasive body contouring procedures. Generally, TFED comprises delivering energy into a confined region inside a body. For convenience hereinafter, the confined region may also be referred to as a treatment area. A transducer element comprised in the transducer unit and adapted to deliver the energy, produces a focused energy beam (TFED beam) whose intensity increases as the beam's cross sectional area decreases towards a focal point in a target area. Intensity may be defined as the energy carried by the TFED beam perpendicularly through unit area per second. At the focal point the area of the focused energy beam is smallest, and intensity is maximal.

Generally, tissue destruction using TFED can be caused by two main mechanisms, namely, thermal and mechanical mechanisms. The thermal mechanism is based on temperature increase within the treatment area. As an acoustic wave propagates through the tissue, part of it is absorbed and converted to heat. When hot enough, which is generally the case in the target area where the focal point is located and beam intensity is greatest, damaging processes may occur, for example the tissue may thermally coagulate. The mechanical mechanism mainly includes streaming, shear forces, tension and inertial cavitation. These processes cause fractionation, rapture and/or liquefaction of cells, which in turn results in tissue destruction.

A number of therapeutic focused energy delivery (TFED) apparatus comprise transducer unit positioning systems including means for tracking a position of the transducer unit. In some cases, the apparatus is adapted to determine the position of the transducer relative to a treatment area and is further adapted to automatically position the transducer unit over the treatment area. Optionally, the apparatus may automatically move the transducer from one treatment area to another. In other cases, a user may view on a display the position of the transducer relative to the treatment area, and may control apparatus movement of the transducer unit over the treatment area by interfacing with the apparatus. Optionally, the user may control apparatus movement from one treatment area to another.

US Patent Application Publication No. 20070232912A1 “Non-invasive Positioning System for Locating the Focus of High-intensity Focused Ultrasound”, describes “a non-invasive positioning system for determining the focus location of a HIFU device comprises a diagnostic ultrasound and the HIFU for ablating and removing tumor tissue. The imaging plane of the diagnostic ultrasound probe and the geometrical axis of a probe of the HIFU define an inclining angle during operation. When the imaging plane of the diagnostic ultrasound intersected to the focus of the HIFU transducer, a maximal convergent interference signal was obtained, so as to position the HIFU focus within tumors for precise ablation.”

US Patent Application Publication No. 20060293598A1 “Motion-tracking Improvements for HIFU Ultrasound Therapy”, describes “high intensity focused ultrasound (HIFU) for medically treating tumors is automatically administered under robotic control in dosage intervals that alternate with ultrasonic imaging intervals. The HIFU transmitter is re-aimed for each dosage to compensate for motion of the tumor due to heart beats and other events.”

U.S. Pat. No. 7,258,674 “Ultrasonic Treatment and Imaging of Adipose Tissue”, describes “a system for the destruction of adipose tissue utilizing high intensity focused ultrasound (HIFU) within a patient's body. The system comprises a controller for data storage and the operation and control of a plurality of elements. One element is a means for mapping a human body to establish three dimensional coordinate position data for existing adipose tissue. The controller is able to identify the plurality of adipose tissue locations on said human body and establish a protocol for the destruction of the adipose tissue. A HIFU transducer assembly having one or more piezoelectric element(s) is used along with at least one sensor wherein the sensor provides feed back information to the controller for the safe operation of the piezoelectric element(s). The sensor is electronically coupled to the controller, and the controller provides essential treatment command information to one or more piezoelectric element(s) based on positioning information obtained from the three dimensional coordinate position data.”

US Patent Application Publication No. 20050154295A1 “Articulating Arm for Medical Procedures”, describes “a two-stage control system being made of a first control means for providing command and control of a robotic arm, and a second control means for the control and movement of a therapy device, such as an ultrasound transducer. The therapy device is positioned within a therapy head. The therapy head is attached to the distal end of the robotic arm. The two-stage control system provides for a macro and micro level of control for the therapy device during a therapy procedure.”

US Patent Application Publication No. 20050187463A1 “Position Tracking Device”, describes “a position tracking device combining one or more optical sensors in a housing with a medical device. The medical device may be an insertion device or one that produces radiant energy. The device may utilize an on-board processor or an external processor to track position data generated by the optical sensors and correlate the treatment regime of the medical device. Alternative embodiments and methods of use are also described.”

### SUMMARY

An aspect of some embodiments of the invention relates to providing a method for performing three dimensional (3D) position-tracking of a transducer unit using two dimensional (2D) optical imaging, and an apparatus adapted to perform the same. The method comprises computing a tilt angle for the transducer unit, using a 2D image acquired by an optical imager when the transducer unit is tilted (roll or pitch, or any combination thereof). The tilt angle of the transducer unit may be defined as an angle between an optical axis of the imager and an axis of a TFED beam transmitted by the transducer unit. Optionally, a tilting movement of the transducer unit may be combined with other transducer unit movements such as yaw and/or lateral displacements in one or more directions, or any combination thereof. For convenience hereinafter, the transducer unit and the optical imager may be referred to as transducer, and imager, respectively. The tilt angle is determined in relation to an optical axis of the optical imager. Computation of the tilt angle may allow for varying the position of the transducer in 3D while the focal point is substantially maintained within a target area.

In the prior art, the transducer comprises a two dimensional (2D) circular target which, when inclined with respect to the optical axis of the imager, is viewed by the imager as elliptical in shape. Based on a known relationship between the shape of the ellipse in a target image, and the circular shape of the 2D target, the tilt angle and the position of the transducer unit in the field of view (FOV) may then be determined. This relationship may be expressed by;

α=ar cos(S/L)

where α is the tilt angle, S is the length of the minor axis in the target image, and L is the length of the diameter of the circular target image (not physically). Nevertheless, a problem of ambiguity arises as the elliptical shape of the target image is substantially the same, for the same tilt angle, when tilting to one direction or the opposite direction.

According to an aspect of some embodiments of the invention, in order to substantially remove ambiguity as to the direction of tilt of the transducer, the transducer comprises a three dimensional (3D) target, for example, a bullseye elevated a height h, relative to a perimeter of the 2D circular target. As the transducer tilt angle increases, the target image acquired by the imager shows a large ellipse associated with the target and a smaller ellipse offset a distance d from the center of the larger ellipse, the smaller ellipse associated with the bullseye. Depending on a direction of tilt, left angle or right angle, the center of the smaller ellipse may be offset towards a left side of the large ellipse, or to a right side, respectively. This offset may be defined as a center offset d, and determines in an unambiguous manner the direction of the tilt angle, resolving the problem of ambiguity. Furthermore in addition to the regular computation of the tilt angle by the eccentricity, computation of a tilt angle α may be made once the target image major axis length L, the smaller ellipse center offset d, and the bullseye height h, are known, and is given by;

α=ar cos(d/L×C), C=h/L.

In some embodiments of the invention, the computations are automatically performed by a position-tracking module comprised by the apparatus. Optionally, the computations may be manually performed by the user, and optionally input to the position-tracking module. Additionally or alternatively, calculation of the center of mass of the large ellipse and the small ellipse may be used to determine d with sub-pixel resolution.

The position-tracking module is adapted to process target images received from the imager and to compare the position of the imaged target with stored data regarding a location, or locations, of one or more treatment areas on a patient's body. Optionally, the location, or locations, of the one or more treatment areas is defined by the user and input to the position-tracking module.

The apparatus may optionally include a transducer motion controller adapted to position the transducer responsive to positioning signals received from the position-tracking module. Optionally, the positioning signals are input by the user. The motion controller is adapted to position the transducer over a treatment area. The motion controller is further adapted to drive the transducer under the constraint of maintaining the focal point substantially within the target area. Positioning the transducer unit may comprise lateral translation in 3 dimensions of space, tilting in any direction in space, and/or rotation

The apparatus additionally comprises a positioning element adapted to move the transducer with 1, 2, 3, 4, 5, or 6 degrees of freedom (up and down, left and right, forward and backward, pitch, yaw, and/or roll, or any combination thereof) while the transducer focal point is maintained within the target area. Optionally, movement of the transducer is automatically performed by the motion controller, responsive to positioning signals received from the position-tracking module, by automatically moving the positioning element, which may be a robot arm, and/or the transducer. Optionally, movement of the transducer is performed by the motion controller, responsive to input signals received from the user, by moving the positioning element, which may be a robot arm, and/or the transducer responsive to the user input signals. Optionally, the positioning element is manually moved by the user. Additionally or alternatively, manual movement of the positioning element is according to data output from the position-tracking module, such as visually displayed data, aural instructions, and the like, or any combination thereof.

In an embodiment of the invention, the transducer may comprise any transducer adapted for TFED and may include a cylindrical shape with a circular target. Optionally, the transducer may have other shapes, for example, quadrilateral, conical, spherical, and the like. The circular target may be a generally flat surface which is marked with one or more concentric circles, for example 2, 3, 4 or more, and an elevated bullseye in the center. Optionally, the target may comprise one or more steps, each adjacent concentric circle at a different height, with the bullseye the most elevated. Optionally, the target may be conically shaped or semi-spherically shaped with the bullseye in a most elevated position, and/or may be transparent with the bullseye marked in the center, or may comprise other markings which allow for distinguishing the bullseye from other markings, or any combination thereof.

In some embodiments of the invention, the imager may comprise a relatively inexpensive camera such as, for example, a CCD (charge coupled device) camera or a CMOS (complementary metal oxide semiconductor) camera.

There is provided, in accordance with an embodiment of the invention, a transducer unit for transmitting focused therapeutic energy, comprising a three dimensional (3D) target, the target adapted to facilitate tracking a tilt angle of the transducer unit. Optionally, the transducer unit is adapted to transmit focused therapeutic energy to a target area tissue of a subject body. Optionally, the focused therapeutic energy comprises ultrasonic energy.

There is provided, in accordance with an embodiment of the invention, a system for facilitating tracking a tilt angle of a therapeutic focused energy transducer unit, the system comprising: a transducer unit comprising a three dimensional (3D) target adapted to facilitate tracking the tilt angle of the transducer unit; and an imager adapted to acquire an image of the target. Optionally, the imager comprises an optical imager. Optionally, the imager is adapted to acquire a two dimensional (2D) image of the target. Optionally, the system further comprises a position-tracking module adapted to track the position of the transducer unit at least partially based on the image of the target obtained by the imager. Optionally, the system is adapted to transmit focused therapeutic energy to a target area tissue of a subject body. Optionally, the focused therapeutic energy comprises ultrasonic energy.

There is provided, in accordance with an embodiment of the invention, a method for facilitating tracking a position of a focused therapeutic energy transducer unit, the method comprising acquiring an image of a three dimensional (3D) target, wherein the three dimensional (3D) target is located on the focused therapeutic energy transducer unit; and tracking a tilt angle of the transducer unit at least partially based on the image. Optionally, the method further comprises acquiring the image with an optical imager. Optionally, the image is a two dimensional (2D) image of the target. Optionally, the method further comprises tracking a position of the transducer unit at least partially based on the image of the target obtained by the imager. Optionally, the method further comprises transmitting focused therapeutic energy to a target area tissue of a subject body. Optionally, the focused therapeutic energy comprises ultrasonic energy.

In some embodiments of the invention, the 3D target is adapted to be separably affixed to the transducer unit. Optionally, the 3D target is an integral part of the transducer unit.

In some embodiments of the invention, the 3D target comprises a recognizable pattern. Optionally, a top view of the 3D target shows a circular shape. Optionally, the 3D target comprises an essentially cylindrically shaped extension. Optionally, the 3D target comprises an essentially conically shaped extension. Optionally, the 3D target is essentially a spherical plane. Optionally, the 3D target comprises at least one concentric circle.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1A schematically illustrates a perspective view of an exemplary transducer, vertically positioned, having a two dimensional (2D) tracking target known in the art;

FIG. 1 B schematically illustrates a top view (as may be seen through a 2D imager) of the 2D tracking target of the exemplary transducer of FIG. 1A;

FIG. 1C schematically illustrates a perspective view of the transducer of FIG. 1A, positioned at a first tilt angle;

FIG. 1D schematically illustrates a top view (as may be seen through a 2D imager) of the 2D tracking target of the exemplary transducer of FIG. 1C;

FIG. 1E schematically illustrates a perspective view of the transducer of FIG. 1A, having a 2D tracking target, positioned at a second tilt angle;

FIG. 1F schematically illustrates a top view of the 2D tracking target of the exemplary transducer of FIG. 1E;

FIG. 2A schematically illustrates a perspective view of an exemplary transducer, vertically positioned, having a three dimensional (3D) tracking target, in accordance with an embodiment of the invention;

FIG. 2B schematically illustrates a top view (as may be seen through a 2D imager) of the 3D tracking target of the exemplary transducer of FIG. 2A, in accordance with an embodiment of the invention;

FIG. 2C schematically illustrates a perspective view of an exemplary transducer having a 3D tracking target, positioned at a first tilt angle, in accordance with an embodiment of the invention;

FIG. 2D schematically illustrates a top view (as may be seen through a 2D imager) of the 3D tracking target of the exemplary transducer of FIG. 2C, in accordance with an embodiment of the invention;

FIG. 2E schematically illustrates a perspective view of an exemplary transducer having a 3D tracking target, positioned at a second tilt angle, in accordance with an embodiment of the invention;

FIG. 2F schematically illustrates a top view (as may be seen through a 2D imager) of the 3D tracking target of the exemplary transducer of FIG. 2E, in accordance with an embodiment of the invention;

FIG. 3A schematically illustrates a perspective view of another exemplary transducer, having a 3D tracking target, positioned at a tilt angle θ, accordance with an embodiment of the invention;

FIG. 3B schematically illustrates a perspective view of another exemplary transducer having a 3D tracking target, positioned at a tilt angle γ, in accordance with an embodiment of the invention;

FIG. 4 schematically illustrates a transducer tracking system, in accordance with an embodiment of the invention; and

FIG. 5 schematically illustrates a flow chart of an exemplary method used by the system shown in FIG. 4, in accordance with an embodiment of the invention.

### DETAILED DESCRIPTION

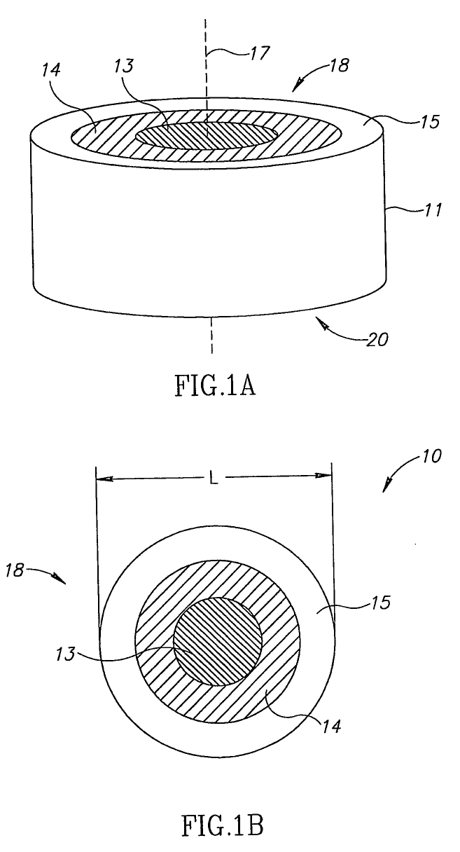

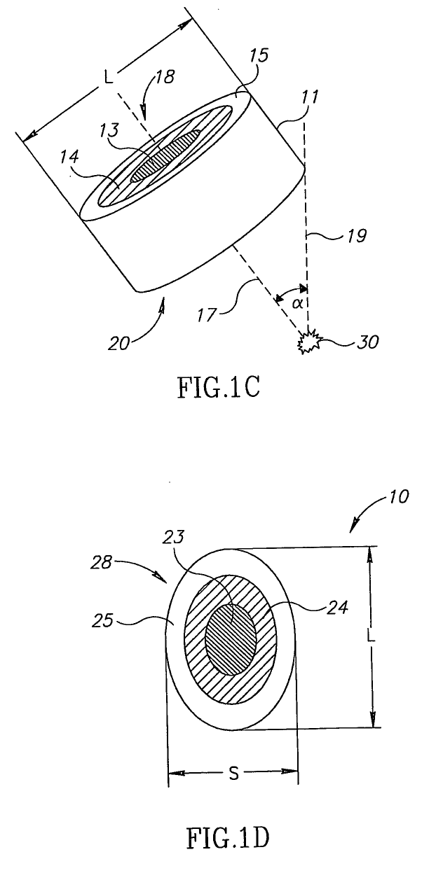

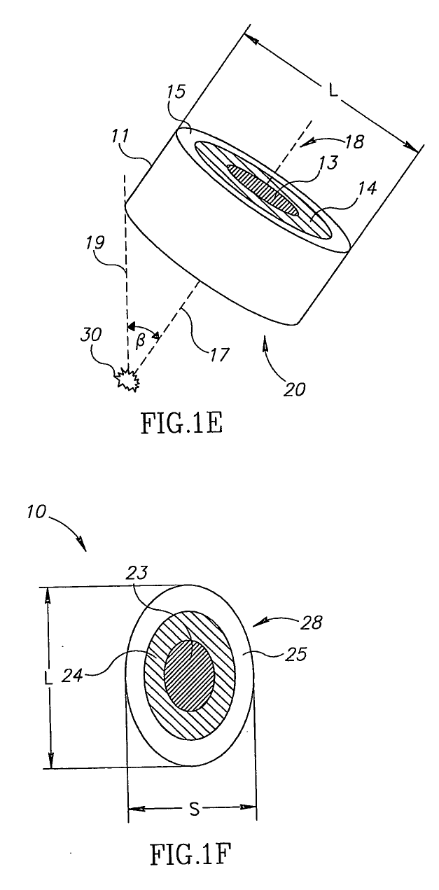

Reference is made to FIG. 1A, which schematically illustrates a perspective view of an exemplary transducer unit 11, vertically positioned, having a two dimensional (2D) tracking target 18, as known in the art. Reference is also made to FIG. 1B which schematically illustrates a top view (as may be seen through a 2D imager 10) of exemplary transducer unit 11 of FIG. 1A, showing 2D tracking target 18.

Transducer unit 11 is adapted to radiate focused therapeutic energy to a treatment area (not shown). Optionally, the radiated focused therapeutic energy may be ultrasound energy. Transducer unit 11 is shown essentially cylindrically shaped.

2D tracking target 18 is located on a top side of transducer unit 11, as shown, opposing a side 20 through which the focused energy is radiated from the transducer unit towards the treatment area. 2D tracking target 18 comprises an outer ring 15, an intermediate ring 14, and a center ring or bullseye 13, all concentrically positioned relative to a center axis 17 in transducer unit 11. Rings 14 and 15, and bullseye 13 are marked such that they may be distinguished from one another when 2D target 18 is viewed through optical imager 10. Center axis 17 extends along a length of transducer unit 11 and coincides with a focal point (not shown) of the transducer.

Reference is made to FIG. 1C, which schematically illustrates a perspective view of transducer unit 11 in FIG. 1A in a first angle tilt, and to FIG. 1D which schematically illustrates 2D tracking target 18 in FIG. 1C, as may be seen from a top view through 2D optical imager 10, as known in the art. The 2D image of 2D target 18 may be referred to hereinafter as target image 28. A first tilt angle α is an angle formed by an intersection of center axis 17, with an optical axis 19 of optical imager 10, at a focal point 30 of transducer unit 11, when the transducer unit is tilted to a side.

Target 18 is positioned on transducer unit 11 such that when optical axis 19 passes through bullseye 13, target image 28 is circular in shape with bullseye 13 substantially centrally located in the target image, for example, similar to that shown in FIG. 1B at 18 and 13 respectively. In this position, center axis 17 substantially coincides with optical axis 19, and tilt angle α is substantially equal to 0 degrees. Otherwise, if optical axis 19 does not pass through bullseye 13, target image 28 is elliptical in shape and comprises elliptically shaped imaged outer ring 25, intermediate ring 24, and bullseye 23. A relationship between the major and minor axis of target image 28 may be used to determine the tilt angle α and the position of the transducer unit in the FOV, as follows:

α=ar cos(S/L)

where α is the tilt angle, S is a length of a minor axis in target image 28, and L is a length of a diameter of circular target image 18.

Reference is made to FIG. 1E, which schematically illustrates a perspective view of transducer unit 11 in FIG. 1A in a second angle tilt, and to FIG. 1F which schematically illustrates 2D tracking target 18 in FIG. 1E, as may be seen from a top view through 2D optical imager 10, as known in the art.

A second tilt angle β is an angle formed by an intersection of center axis 17, with optical axis 19. Target 18 is positioned on transducer unit 11 such that when optical axis 19 passes through bullseye 13, target image 28 is circular in shape, with bullseye 13 substantially centrally located in the target image, for example, similar to that shown in FIG. 1B at 18 and 13 respectively. In this position, center axis 17 substantially coincides with optical axis 19, and tilt angle β is substantially equal to 0 degrees. Otherwise, if optical axis 19 does not pass through bullseye 13, target image 28 is elliptical in shape and comprises an elliptically shaped imaged outer ring 25, intermediate ring 24, and bullseye 23.

A problem of ambiguity arises when α=″β as target image 28 is substantially the same, regardless of whether transducer 11 is tilted to first angle α or to second angle β. Consequently, it is usually not possible to determine in which direction transducer unit 11 is tilted.

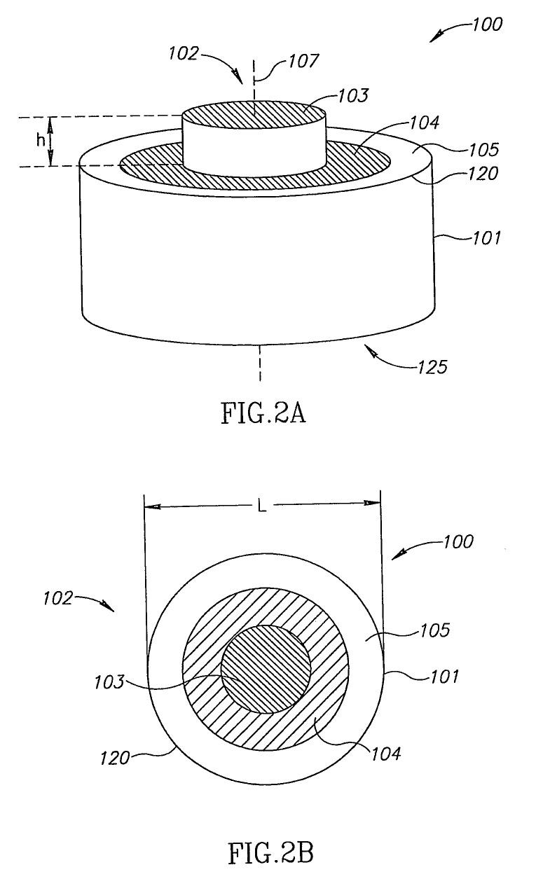

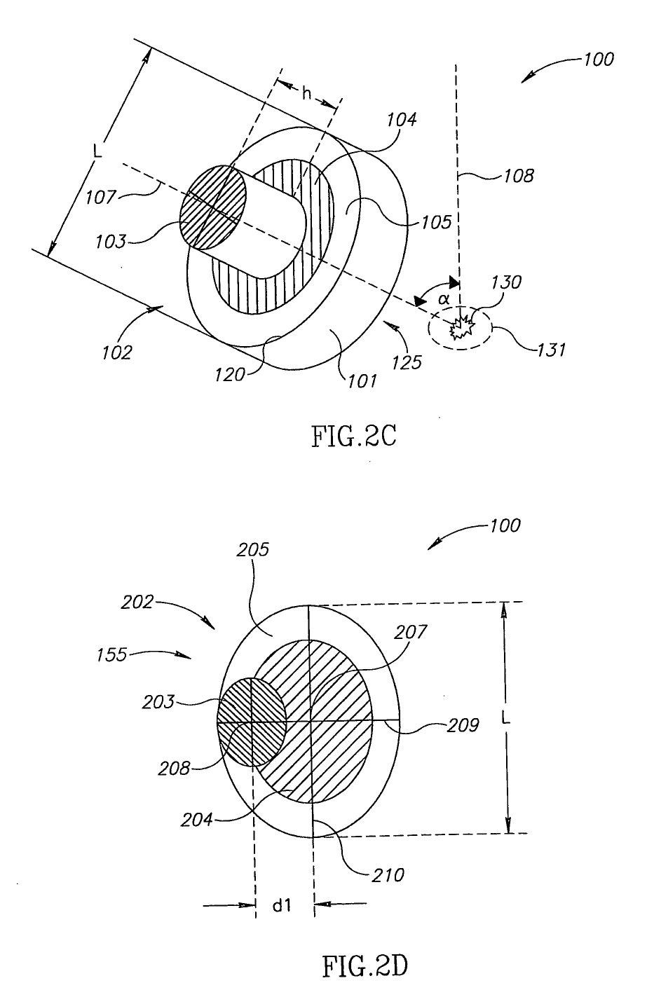

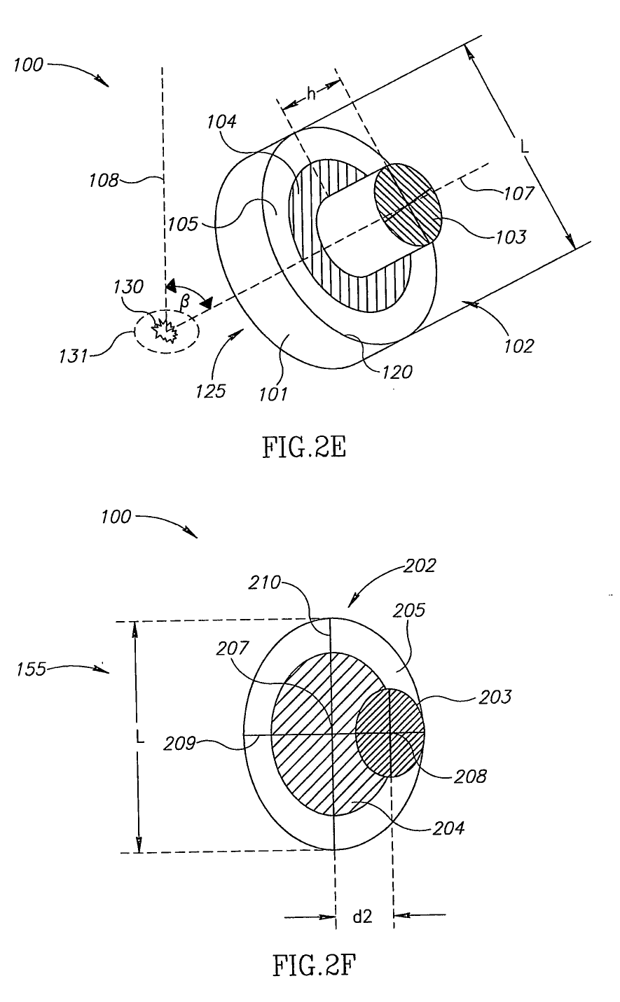

Reference is made to FIG. 2A, which schematically illustrates a perspective view of an exemplary transducer unit 101 comprised in a TFED apparatus 100, and to FIG. 2B which schematically illustrates an exemplary three-dimensional (3D) target 102 comprised in transducer unit 101 in FIG. 2A, in accordance with an embodiment of the invention. Transducer unit 101 is adapted to radiate focused therapeutic energy to a treatment area (not shown). Optionally, the radiated focused therapeutic energy may be ultrasound energy. Transducer unit 101 is shown essentially cylindrically shaped, and may optionally have other shapes, such as, for example, quadrilateral, conical, semi-spherical, and the like.

Target 102 is located on a top side of transducer unit 101, as shown, opposing a side 125 through which the focused energy is radiated from the transducer unit towards a treatment area (not shown). Target 102 comprises an outer ring 105, an intermediate ring 104, and an elevated center ring or bullseye 103, all concentrically positioned relative to a center axis 107 in transducer unit 101. Rings 104 and 105, and bullseye 103 are marked such that they may be distinguished from one another when target 102 is viewed through an optical imager (not shown). Optionally, target 102 may comprise only outer ring 105 and elevated bullseye 103. Optionally, target 102 may comprise additional rings, for example 3, 4, or 5 rings. Additionally or alternatively, rings 104 and 105 may be at different heights (steps). Center axis 107 extends along a length of transducer unit 101 and coincides with a focal point (not shown) of the transducer.

In accordance with an embodiment of the invention, bullseye 103 is cylindrically shaped with a flat top, and is elevated a height h above an outer perimeter 120 comprised in outer ring 105, the elevated bullseye providing for a 3D target 102. Optionally, bullseye 103 may be conically shaped, dome shaped, cylindrically shaped with a conical top, cylindrically shaped with a round top, or any combination thereof, a height h above outer perimeter 120 providing for a 3D target 102. Optionally, bullseye 103 is elevated above all rings in target 102.

Reference is made to FIG. 2C, which schematically illustrates a perspective view of transducer unit 101 in FIG. 2A in a first angle tilt, and to FIG. 2D which schematically illustrates target 102 in FIG. 2C as may be seen through a 2D optical imager 155, also comprised in TFED apparatus 100, in accordance with an embodiment of the invention.

In accordance with an embodiment, a first tilt angle α is computed for transducer unit 101 using a 2D image of target 102, as acquired by optical imager 155. The 2D image of target 102 may be referred to hereinafter as target image 202. The first tilt angle α is an angle formed by an intersection of center axis 107, with an optical axis 108 of optical imager 155, at transducer unit 101 focal point 130, when transducer unit 101 is tilted to a side. Computation of tilt angle α may allow for varying the position of transducer unit 101 over the treatment area while focal point 130 is maintained within a target area 131 in the treatment area.

Target 102 is positioned on transducer unit 101 such that when optical axis 108 passes through a center of the elevated target, for example through bullseye 103, target image 202 is substantially circular in shape, with bullseye 203 substantially centrally located in the target image, for example, similar to that shown in FIG. 1B at 102 and 103 respectively. In this position, center axis 107 substantially coincides with optical axis 108, and tilt angle α is substantially equal to 0 degrees. Otherwise, if optical axis 108 does not pass through bullseye 103, target image 202 is elliptical in shape and comprises an elliptically shaped imaged outer ring 205, intermediate ring 204, and bullseye 203. Target image 202 may further comprise a minor axis 209, a major axis 210 and a target image center 207, defined by the intersection of the major axis and the minor axis.

Bullseye 103 is elevated a height h, relative to outer perimeter 120. Depending on a direction of tilt, for example, the first angle tilt as shown by angle α, imaged bullseye 203 may be offset towards a left side of major axis 210 along minor axis 209. This offset may be measured by a center offset d1 between an imaged bullseye center 208 and target image center 207. As tilt angle α increases, center offset d1 increases; and inversely, as tilt angle α decreases, center offset d1 decreases. Computation of the tilt angle α may be made once the circular target 102 length L, the smaller elliptical bullseye 203 center offset d1, and bullseye 103 height h, are known, and is given by;

α=ar cos(d1/L×C), C=h/L.

Reference is also made to FIG. 2E which schematically illustrates a perspective view of transducer unit 101 in FIG. 2A in a second angle tilt, and to FIG. 2F which schematically illustrates target 102 in FIG. 2E as may be seen through 2D optical imager 155, in accordance with an embodiment of the invention. A second tilt angle β is an angle formed by an intersection of center axis 107 with optical axis 108, at focal point 130, when transducer unit 101 is tilted in a direction relatively opposite to the first angle tilt. For a second angle tilt as shown by angle β, imaged bullseye 203 may be offset towards a right side of major axis 210 along minor axis 209. This offset may be measured by a center offset d2 between imaged bullseye center 208 and target image center 207. As tilt angle β increases, center offset d2 increases; and inversely, as tilt angle β decreases, center offset d2 decreases. Computation of the tilt angle β may be made once the circular target 102 length L, the smaller elliptical bullseye 203 center offset d2, and bullseye 103 height h, are known, and is given by;

β=ar cos(d2/L×C), C=h/L.

In accordance with an embodiment of the invention, symmetry in the circular shape of target 102 allows for transducer unit 101 to be tilted in any direction at an angle α, or β, and for the center offset d1, or d2, to be measured as a displacement along minor axis 209 of imaged bullseye 203 either to the left or right of major axis 210. Computation of the tilt angle α or β may be made once the length of the major axis, L, the bullseye center offset d1 or d2, and the bullseye height h, are known. Optionally, calculation of the center of mass of the large ellipse and the small ellipse may be used to determined.

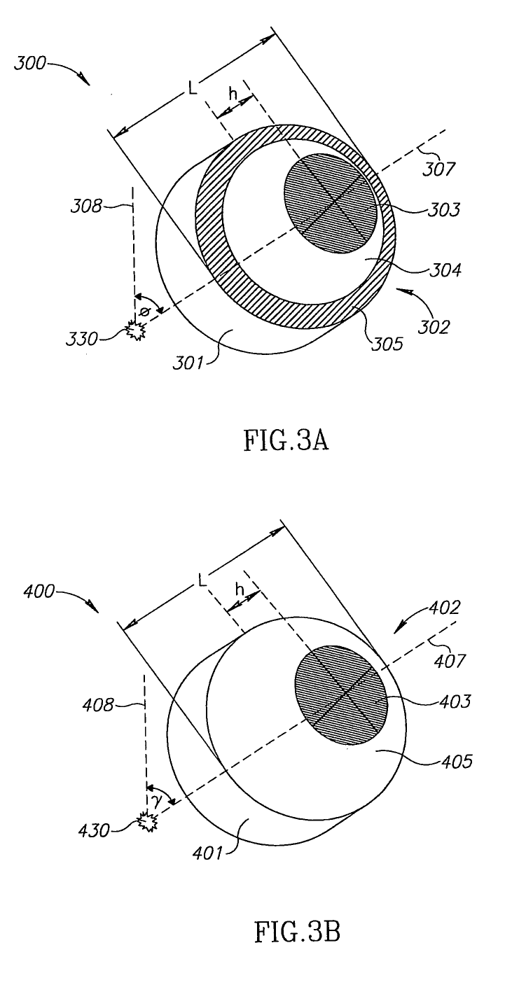

Reference is made to FIG. 3A, which schematically illustrates a perspective view of an exemplary transducer unit 301 comprised in a TFED apparatus 300, the transducer unit comprising a 3D elevated target 302, in accordance with another embodiment of the invention. Transducer unit 301 is tilted such that a center axis 307 intersects optical axis 308 at an angle θ at transducer 301 focal point 330. Apparatus 300, including transducer unit 301, center axis 307, and optical axis 308 may be the same or substantially similar to that shown in FIGS. 2A, 2C and/or 2E at 100, 101, 107 and 108 respectively, with the exception that elevated target 302 is dome shaped.

Target 302 comprises an outer ring 305, an intermediate ring 304, and a center ring or bullseye 303, all concentrically positioned relative to center axis 307. Rings 304 and 305, and bullseye 303 are marked such that they may be distinguished from one another when target 302 is viewed through an optical imager (not shown). Optionally, target 302 may comprise only outer ring 305 and bullseye 303. Optionally, target 302 may comprise additional rings, for example 3, 4, or 5 rings. A height of target 302 is given by h. Computation of the tilt angle θ may be made once circular target 302 length L, the smaller elliptical bullseye (not shown) center offset d, and bullseye 303 height h, are known, and is given by;

θ=ar cos(d/L×C), C=h/L.

Reference is made to FIG. 3B, which schematically illustrates a perspective view of an exemplary transducer unit 401 comprised in a TFED apparatus 400, the transducer unit comprising a 3D elevated target 402, in accordance with another embodiment of the invention. Transducer unit 401 is tilted such that a center axis 407 intersects optical axis 408 at an angle γ at transducer 401 focal point 430. Apparatus 400, including transducer unit 401, center axis 407, and optical axis 408 may be the same or substantially similar to that shown in FIGS. 2A, 2C and/or 2E at 100, 101, 107 and 108 respectively, with the exception that elevated target 402 is dome shaped and comprises a transparent outer ring 405 and a bullseye 403. Transparent outer ring 405 and bullseye 403 are concentrically positioned relative to center axis 407. Bullseye 403 is marked such that it may be distinguished when target 402 is viewed through an optical imager (not shown). A height of target 402 is given by h. Computation of the tilt angle γ may be made once circular target 402 length L, the smaller elliptical bullseye (not shown) center offset d, and bullseye 403 height h, are known, and is given by;

γ=ar cos(d/L×C), C=h/L.

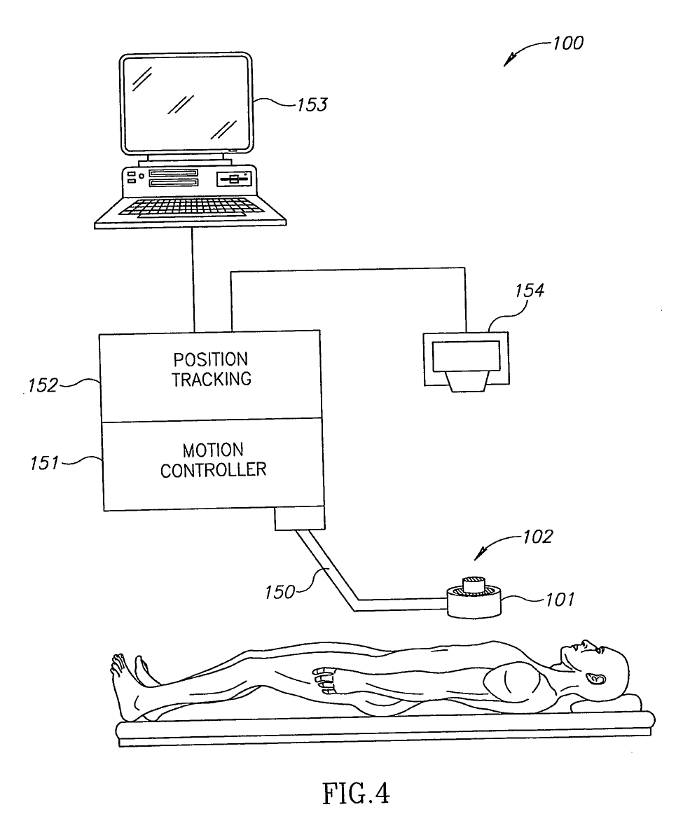

Reference is made to FIG. 4, which schematically illustrates TFED apparatus 100 shown in FIGS. 2A, 2B, 2C, 2D, 2E, and 2F, in accordance with an embodiment of the invention. Apparatus 100 comprises transducer unit 101, including target 102, a position element 150, an optional motion controller 151, a position-tracking module 152, a user input/out (I/O) interface module 153, and an optical imager 154. In accordance with an embodiment of the invention, apparatus 100 is adapted to perform 3D position-tracking of transducer unit 101 using 2D optical imaging. Apparatus 100 may optionally be adapted to automatically position transducer unit 101 over a treatment area (not shown). Optionally, apparatus 100 may be adapted to automatically move transducer unit 101 from a first treatment area to a second treatment area, and may be further optionally adapted to vary the position of transducer unit 101 over the treatment area, while maintaining focus on a focal point (not shown).

Optical imager 154 is adapted to acquire one or more 2D images of the treatment area and/or target 102, and to transmit target images to position-tracking module 152 for processing, including optional computation of the tilt angle. Optical imager 154 is further adapted to provide real-time 2D images of target 102 over the treatment area which, when processed by position tracking module 152, allow for real-time tracking of the position of transducer unit 101 over one or more treatment areas. Optionally, imager 154 may comprise a display for displaying target images and images acquired by the imager. Optionally, imager 154 may be adapted to interface with I/O interface module 153, and may be further adapted to transfer images directly to the interface module. Imager 154 may comprise a relatively inexpensive camera such as, for example a CCD camera or a CMOS camera.

Position-tracking module 152 is adapted to process target images received from imager 154 and is further adapted to compare the position of one or more target images with stored data regarding a location, or locations, of one or more treatment areas on a patient's body (not shown). Optionally, the location, or locations, of the one or more treatment areas is defined by the user and input to position-tracking module 152 by means of, for example, I/O interface module 153. Position-tracking module 152 is further adapted to compute the tilt angle of transducer unit 101. Optionally, the computations may be manually performed by the user and, optionally, input to position-tracking module 152. Optionally, processed data by position-tracking module 152 may be provided to the user by means of, for example, I/O interface module 153. Optionally, the processed data may include target images.

Transducer motion controller 151 is adapted to position transducer unit 101, responsive to positioning signals received from position-tracking module 152. Optionally, the positioning signals are input by the user. Motion controller 151 is adapted to position transducer unit 101 over a treatment area. Motion controller 151 is further adapted to drive transducer unit 101 under the constraint of maintaining the focal point substantially within a target area (not shown). Positioning transducer unit 101 may comprise lateral translation in 3 dimensions of space, tilting in any direction in space, and/or rotation.

Positioning element 150 is adapted to move transducer unit 101 with 1, 2, 3, 4, 5, or 6 degrees of freedom (up and down, left and right, forward and backward, pitch, yaw, and/or roll, or any combination thereof) while the focal point is maintained within the target area. Optionally, movement of transducer unit 101 is automatically performed by motion controller 151, responsive to positioning signals received from position-tracking module 152, by automatically moving positioning element 150, which may be a robot arm, and/or the transducer. Optionally, movement of transducer unit 101 is performed by motion controller 151, responsive to input signals received from the user, by moving positioning element 150, which may be a robot arm, and/or the transducer unit responsive to the user input signals. Optionally, positioning element 150 is manually moved by the user. Additionally or alternatively, manual movement of positioning element 150 is according to data output from position-tracking module 152, such as visually displayed data, aural instructions, and the like, or any combination thereof.

I/O interface module 153 is adapted to allow the user input/output data interfacing with apparatus 100. I/O interface module 153 may comprise data output means such as, for example: a display, loudspeakers, printer, and the like. Information displayed may include, for example, target images; tilt angles; tilt angle calculation parameter such as length L of the major axis, height h of the bullseye, and central offset d1 or d2; position of target 102 over treatment area; close up images of treatment area; general view of all treatment areas; data input by user; and the like. I/O interface module 153 may additionally comprise input means such as a keyboard, a mouse, a scanner, and the like. Information input may include, for example, details regarding the number, size and location of treatment areas; type of transducer; height h of bullseye; and the like. Additionally comprised in I/O interface module 153 may be data storage means, and/or optional data communications means, including that for wired and/or wireless communications.

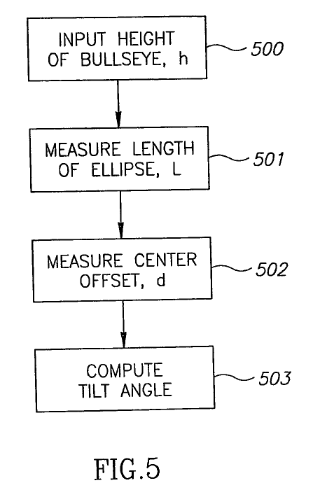

Reference is made to FIG. 5, which schematically illustrates a flow chart of an exemplary method used by apparatus 100, shown in FIG. 4, to calculate a tilt angle for transducer unit 101 when performing 3D tracking of the position of the transducer, in accordance with an embodiment of the invention. The method described is not intended to be limiting, and it may be clear to a person skilled in the art that other operations, combinations and/or sequences of steps may be used when operating apparatus 100.

[STEP 500] Input a height of a bullseye comprised in target 102. For example, the height may be input by the user through I/O interface module 153, and/or, the height may be previously stored in apparatus 100, and may be automatically retrieved by the apparatus when transducer unit 101 and/or target 102 are identified.

[STEP 501] Imager 154 acquires images of target area 102. Position-tracking module 152 processes the target images and computes a length L of the major axis. Optionally, the target images may be displayed on a display comprised in I/O interface module 153. Optionally, imager 154 comprises a display where the target image is displayed. The length L may be measured by a user.

[STEP 502] Position-tracking module 152 processes the target images and computes a center offset d1, or d2, of the bullseye along the minor axis. Optionally, the user may measure the center offset from the display on I/O interface module 153 and/or, the optional display on imager 154.

[STEP 503] Position-tracking module 152 computes the tilt angle of transducer unit 101 and displays the tilt angle on I/O interface module 153. Optionally, the user calculates the tilt angle, and optionally inputs the tilt angle to position-tracking module 152 by means of I/O interface module 153.

In some embodiments of the invention, the computations are automatically performed by a position-tracking module comprised by the apparatus. Optionally, the computations may be manually performed by the user, and optionally input to the position-tracking module.

In the description and claims of embodiments of the present invention, each of the words, “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art.

1. A transducer unit for transmitting focused therapeutic energy comprising:
a three dimensional (3D) target, the target adapted to facilitate tracking a tilt angle of the transducer unit.

2. The transducer unit of claim 1 wherein the 3D target comprises a recognizable pattern.

3. The transducer unit of claim 1, wherein a top view of the 3D target shows a circular shape.

4. The transducer unit of claim 1, wherein the 3D target comprises an essentially cylindrically shaped extension.

5. The transducer unit of claim 1, wherein the 3D target comprises an essentially conically shaped extension.

6. The transducer unit of claim 1, wherein the 3D target is essentially a spherical plane.

7. The transducer unit of claim 1, wherein the 3D target comprises at least one concentric circle.

8. The transducer unit of claim 1, wherein the 3D target is adapted to be separably affixed to the transducer unit.

9. The transducer unit of claim 1, wherein the 3D target is an integral part of the transducer unit.

10. The transducer unit of claim 1, adapted to transmit focused therapeutic energy to a target area tissue of a subject body.

11. The transducer unit of claim 10, wherein the focused therapeutic energy comprises ultrasonic energy.

12. A system for facilitating tracking a tilt angle of a therapeutic focused energy transducer unit, the system comprising:
a transducer unit comprising a three dimensional (3D) target adapted to facilitate tracking the tilt angle of the transducer unit; and
an imager adapted to acquire an image of the target.

13. The system of claim 12, wherein the imager comprises an optical imager.

14. The system of claim 12, wherein the imager is adapted to acquire a two dimensional (2D) image of the target.

15. The system of claim 12, further comprising a position-tracking module adapted to track the position of the transducer unit at least partially based on the image of the target obtained by the imager.

16. The system of claim 12 wherein the 3D target comprises a recognizable pattern.

17. The system of claim 12, wherein a top view of the 3D target shows a circular shape.

18. The system of claim 12, wherein the 3D target comprises an essentially cylindrically shaped extension.

19. The system of claim 12, wherein the 3D target comprises an essentially conically shaped extension.

20. The system of claim 12, wherein the 3D target is essentially a spherical plane.

21. The system of claim 12, wherein the 3D target comprises at least one concentric circle.

22. The system of claim 12, wherein the 3D target is adapted to be separably affixed to the transducer unit.

23. The system of claim 12, wherein the 3D target is an integral part of the transducer unit.

24. The system of claim 12, adapted to transmit focused therapeutic energy to a target area tissue of a subject body.

25. The system of claim 24, wherein the focused therapeutic energy comprises ultrasonic energy.

26. A method for facilitating tracking a position of a focused therapeutic energy transducer unit, the method comprising:
acquiring an image of a three dimensional (3D) target, wherein the three dimensional (3D) target is located on the focused therapeutic energy transducer unit; and
tracking the tilt angle of the transducer unit at least partially based on the image.

27. The method of claim 26 further comprising acquiring the image with an optical imager.

28. The method of claim 26 wherein the image is a two dimensional (2D) image of the target.

29. The method of claim 26 further comprising tracking a position of the transducer unit at least partially based on the image of the target obtained by the imager.

30. The method of claim 26 wherein the 3D target comprises a recognizable pattern.

31. The method of claim 26 wherein a top view of the 3D target shows a circular shape.

32. The method of claim 26 wherein the 3D target comprises an essentially cylindrically shaped extension.

33. The method of claim 26 wherein the 3D target comprises an essentially conically shaped extension.

34. The method of claim 26 wherein the 3D target is essentially spherical plane.

35. The method of claim 26 further comprising concentrically locating at least one circle within the 3D target.

36. The method of claim 26 further comprising separably affixing the target to the transducer unit.

37. The method of claim 26 wherein the 3D target is an integral part of the transducer unit.

38. The method of claim 26 further comprising transmitting focused therapeutic energy to a target area tissue of a subject body.

39. The method of claim 38, wherein the focused therapeutic energy comprises ultrasonic energy.
